(19) United States
(12) Patent Application Publication
(10) Pub. No.: US 2024/0052285 A1
(43) Pub. Date: Feb. 15, 2024

(54) IMAGING-ENABLED BIOREACTOR FOR IN VITRO CULTIVATION AND BIOENGINEERING OF ISOLATED AIRWAY TISSUE

(71) Applicant: THE TRUSTEES OF THE STEVENS INSTITUTE OF TECHNOLOGY, Hoboken, NJ (US)

(72) Inventors: Jinho KIM, Jersey City, NJ (US); Seyed Mohammad MIR, Hoboken, NJ (US); Jiawen CHEN, Hoboken, NJ (US)

(73) Assignee: THE TRUSTEES OF THE STEVENS INSTITUTE OF TECHNOLOGY, Hoboken, NJ (US)

(21) Appl. No.: 18/234,261

(22) Filed: Aug. 15, 2023

Related U.S. Application Data
(60) Provisional application No. 63/398,198, filed on Aug. 15, 2022.

(51) Int. Cl. C12M 1/34; C12M 3/06

(52) U.S. Cl. CPC C12M 41/00; C12M 27/16

(57) ABSTRACT

Systems and methods associated with a bioreactor are disclosed. An imaging module is provided that allows for in situ observation of, for instance, lung tissue. Various compounds can be introduced into a cell culture chamber for experimental and practical applications on epithelial tissue. Methods and apparatus are also provided for deepithelializing human or rat tissue without damaging the structures underneath. Thereafter, cell-growth can be effected in a homogenous distribution.

### CROSS-REFERENCE TO RELATED APPLICATION

This application claims priority to U.S. Provisional Patent Application Ser. No. 63/398,198 filed Aug. 15, 2022, the entire disclosure of which, including any and all Appendices, is incorporated herein by reference.

### STATEMENT REGARDING FEDERALLY SPONSORED RESEARCH

To be determined.

### FIELD OF THE INVENTION

The invention relates to in-situ imaging modalities to allow in vitro cultivation tissue reconstruction, real-time visualization of isolated airway tissues, mechanically assisted ablation and cell seeding.

### BACKGROUND OF THE INVENTION

The human conducting airways are lined by the airway epithelium that mostly consists of multi-ciliated, club, goblet, and basal cells. These airway epithelial cells collectively create a protective biophysical barrier between the external environment and underlying tissues against inhaled harmful substances, such as pathogens, allergens, chemical gases, or particulates. The protective functions of the airway epithelium include mucociliary clearance, tight junction formation, and antimicrobial secretion. In addition to serving as the first line of defense of the lung, the airway epithelium is the prime site for the initiation and progression of many devastating respiratory disorders. To understand pathophysiology of airway diseases and develop therapeutics in a cost-effective manner, different types of in vitro airway tissue models have been created. In particular, decellularized airway tissues have been used to investigate survival, proliferation, and differentiation of both healthy and diseased airway cells by providing tissue-specific microenvironments to implanted airway cells.

### SUMMARY OF THE INVENTION

The invention encompasses a bioreactor system integrated with an in-situ imaging modality that can allow in vitro cultivation, tissue reconstruction, and real-time visualization of isolated airway tissues, such as rat trachea or human small airways (see FIG. 1 and FIG. 2), as well as a method for mechanically assisted ablation of the endogenous epithelium from the in vitro cultured airway tissue (FIG. 3A) followed by homogeneous and rapid distribution of exogenous epithelial cells via hydrogel-based cell seeding topically onto the deepithelialized airway lumen (FIG. 3B).

The imaging-enabled bioreactor system can serve as a platform to culture a small segment of airway tissue while the interior of the airway tissue can be visualized at the cellular level. When this system is coupled with a novel epithelium replacement method, the mechanically assisted removal of endogenous airway epithelial layer and subsequent topical implantation of lab-grown epithelial cells for rapid regeneration of functional airway epithelium are facilitated.

The imaging-enabled bioreactor allows for in vitro cultivation of isolated small airway tissues (e.g., rat tracheas or human small airways) and provide the following advantages.

It is an object of the present invention to enable real-time assessment of the airway tissue. Existing commercially available bioreactor platforms for cultivating hollow organs (e.g., trachea, bronchi, blood vessels) include, for example, InBreath 3D Bioreactor (Harvard Apparatus) and have been used for seeding and culturing of different cell types on either side of the tubular tissue. However, existing bioreactor platforms lack in real-time imaging capability. Thus, evaluation of tissue structure and function can be achieved only after removing and processing tissue samples from the cultivated tissue. In contrast, the present optical fiber-based airway imaging modality enables both fluorescent and bright-field visualization of the cells and the tissue in situ in real time. This real-time and rapid tissue monitoring capability can allow non-destructive rapid structural and functional evaluation of the cultured tissues.

It is another object of the present invention to facilitate uniform distribution of newly implanted cells on the airway lumen. Current cell delivery methods use culture medium to deliver the cells into the luminal surface of the trachea. This causes the inhomogeneous distribution and accumulation of cells on the lower surface of the airway tissues mainly due to gravity. This invention allows for the homogenous distribution of cells on the entire lumen that provides enormous advantages in the creation of functional in vitro airway tissues.

It is a further object of the present invention to enable real-time direct visualization of the airway tissue at cellular level. Currently, microscopic assessments of the cultured cells are only possible after removing the cell-tissue constructs upon completion of each experiment. However, the presently proposed optical fiber imaging modality integrated with the bioreactor allows for real-time monitoring of the lumen of ex vivo airway tissues during controlled cell removal, seeding, and ex vivo culture.

It is yet another object of the present invention to promote preservation of native airway tissue components. The established protocols for airway decellularization are based on exposing the airway tissue to a strong decellularization agent under harsh conditions (e.g., high pH) to remove all cellular components from the tissue. This reduces the number of collagen fibers, glycosaminoglycans (GAGs), proteoglycans, and chondrocytes, compromising the biochemical and mechanical properties of the airway tissue. However, the platform of the present invention allows for selective removal of the epithelial layer from the airway lumen without disrupting the underlying tissue layers and extracellular matrix.

It is a not necessarily final object of the present invention to promote rapid regeneration of functional airway epithelium. To promote reconstruction of fully functional airway epithelium, the implanted new cells need to be distributed uniformly and rapidly, and adhered persistently across the de-epithelialized lumen of the airway tissue during their proliferation and differentiation. The inventive platform provides rapid coverage of the de-epithelialized airway surface that can maximize regeneration of functional epithelium with reduced risk for contamination of the tissue constructs.

### Other Possible Commercial Applications of the Present Invention Include:

Lung-on-a-chip devices, which have been developed to mimic pathophysiology of human lung on a microchip to study human lung diseases. The inventive device can serve as an airway-on-chip platform to model various airway diseases and study the mechanisms of airway-specific diseases.

The invention can also be used to create in vitro-cultured airway tissues to test drug candidates for different airway diseases, such as cystic fibrosis, primary ciliary dyskinesia, asthma, and COPD.

The system and method for selective removal followed by topical implantation of the airway epithelial cells can be applied to other organs or tissues with a tubular configuration (e.g., intestine, ovary, esophagus, bladder, etc.) to create their in vitro counterparts.

The cell removal and subsequent cell seeding method can be used to repair diseased or damaged airway tissue via localized cell replacement.

In summary, the present invention addresses the lack of commercial devices to culture small airway tissues and monitor the tissue at cellular level in real time. Moreover, using this device, humanized airway tissue can be generated by selectively removing epithelium from a rat trachea and implanting human stem cells/epithelial cells. The device also addresses the lack of a protocol for homogenous distribution of cells in the airway lumen.

### BRIEF DESCRIPTION OF THE DRAWINGS

For a more complete understanding of the present invention, reference is made to the following detailed description of various embodiments considered in conjunction with the accompanying drawings, in which:

FIG. 1 is a schematic overview of an imaging-enabled bioreactor and de-epithelialization of ex vivo rat trachea in accordance with an embodiment of the present invention;

FIG. 2 is a schematic showing an imaging probe being used for visual inspection of an in vitro-cultured airway tissue in accordance with an embodiment of the present invention—TL: tube lens. F: optical filter. DM: dichroic mirror. OL: objective lens. IP: imaging probe;

FIG. 3 is a schematic overview of the epithelium removal and cell implantation processes in accordance with an embodiment of the present invention;

FIG. 4 is a schematic illustration and associated charts illustrating mechanical vibration for the clearance of detergent-disrupted epithelial cells in accordance with an embodiment of the present invention;

FIG. 5 is a series of images illustrating in situ visualization of the trachea lumen using a custom-built micro-optical imaging device in accordance with an embodiment of the present invention;

FIG. 6 is a series of images and associated graphs illustrating the histological analysis of ECM microstructure and components of native and de-epithelialized trachea in accordance with an embodiment of the present invention;

FIG. 7 is a series of images and associated graphs illustrating the evaluation of ECM components via immunohistochemistry and DNA/GAG quantification in accordance with an embodiment of the present invention;

FIG. 8 is a series of SEM images depicting the luminal surfaces of de-epithelialized tracheas;

FIG. 9 is a series of SEM images illustrating the rat trachea lumen treated with 2% and 4% SDS in the absence of mechanical vibration in accordance with an embodiment of the present invention;

FIG. 10 is a series of images and associated graphs illustrating the topical deposition of exogenous cells onto de-epithelialized rat tracheal lumen in accordance with an embodiment of the present invention; and

FIG. 11 is a series of images and associated graphs illustrating the proliferation of seeded mesenchymal stem cells on the de-epithelialized rat tracheal lumen in accordance with an embodiment of the present invention.

### DETAILED DESCRIPTION OF THE EXEMPLARY EMBODIMENT

The following disclosure is presented to provide an illustration of the general principles of the present invention and is not meant to limit, in any way, the inventive concepts contained herein. Moreover, the particular features described in this section can be used in combination with the other described features in each of the multitude of possible permutations and combinations contained herein.

All terms defined herein should be afforded their broadest possible interpretation, including any implied meanings as dictated by a reading of the specification as well as any words that a person having skill in the art and/or a dictionary, treatise, or similar authority would assign thereto.

Further, it should be noted that, as recited herein, the singular forms “a”, “an”, “the”, and “one” include the plural referents unless otherwise stated. Additionally, the terms “comprises” and “comprising” when used herein specify that certain features are present in that embodiment, however, this phrase should not be interpreted to preclude the presence or addition of additional steps, operations, features, components, and/or groups thereof.

All examples and conditional language recited herein are intended for pedagogical purposes to aid the reader in understanding the principles of the invention and the concepts contributed by the inventor to furthering the art, and are to be construed as being without limitation to such specifically recited examples and conditions. Moreover, all statements herein reciting principles, aspects, and embodiments of the invention, as well as specific examples thereof, are intended to encompass both structural and functional equivalents thereof. Additionally, it is intended that such equivalents include both currently known equivalents as well as equivalents developed in the future, i.e., any elements developed that perform the same function, regardless of structure.

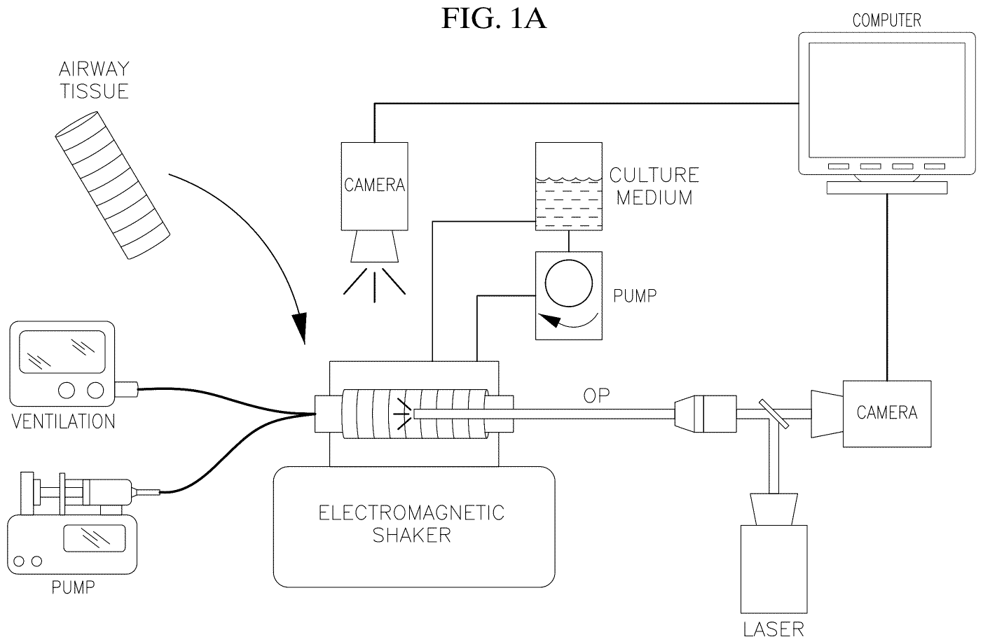

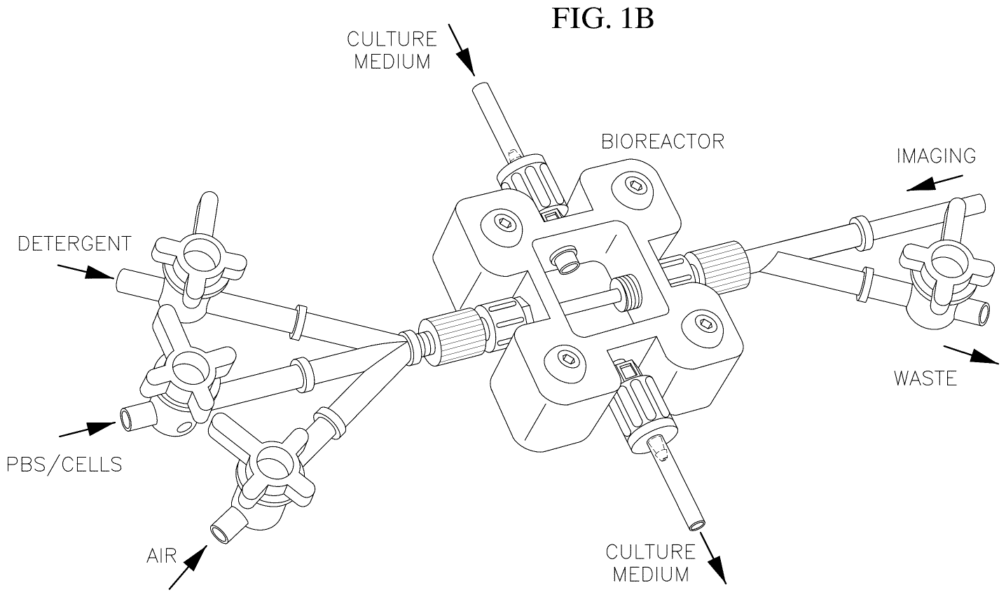

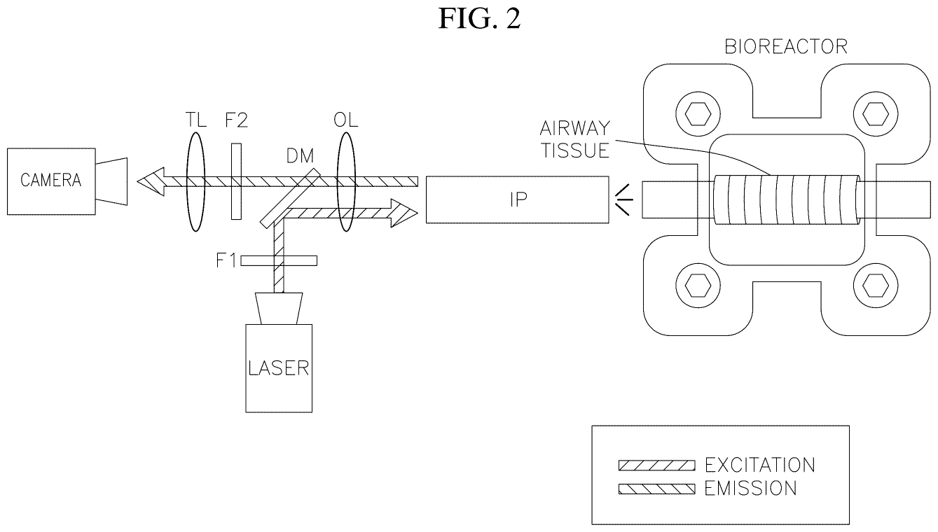

The invention encompasses a device that can be used to (i) apply user-defined biochemical treatments to ex vivo airway tissue; (ii) real-time monitoring of the interior and exterior surfaces of the airway at the cellular level; (iii) uniformly distribute newly implanted cells; and (iv) long-term culturing of ex vivo tissues following topical seeding of exogeneous cells. The device includes i) a custom-built bioreactor with airway culture chamber in which the airway tissue can be placed for de-epithelialization and subsequent ex vivo culture (FIGS. 1A, 1B) and ii) an optical fiber imaging element that allows real-time monitoring of the interior and exterior surfaces of the airway at the cellular level (FIG. 2).

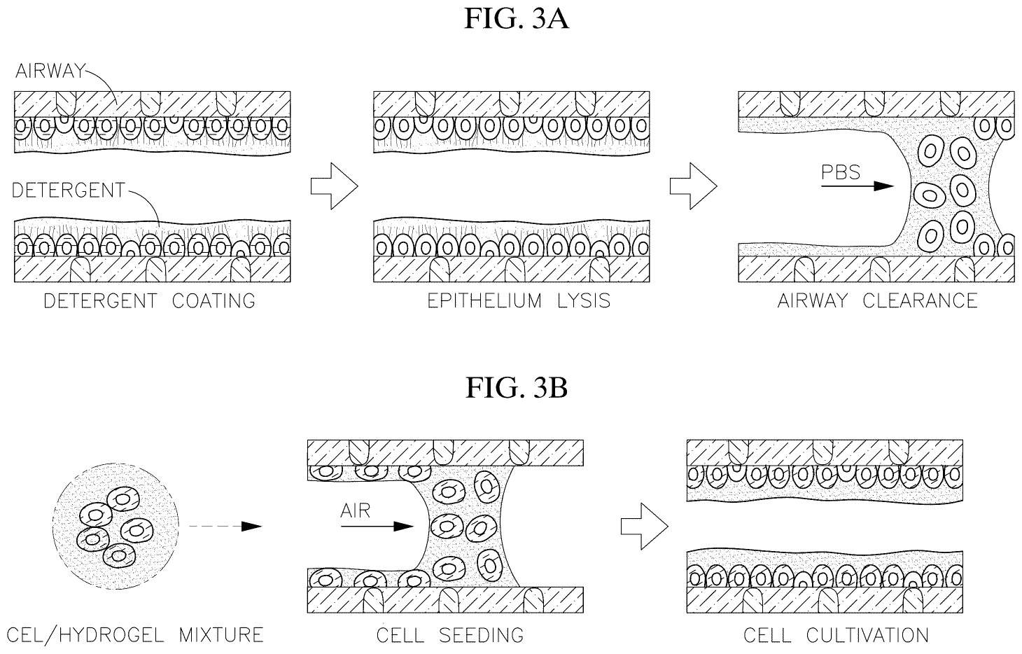

Moreover, a device made in accordance with an embodiment of the present invention can be used to develop i) a protocol that allows selective removal of the endogenous epithelium of in vitro-cultured airway tissues and preservation of the airway extracellular matrix (FIG. 3A) and ii) a hydrogel-based cell delivery method for uniform distribution of newly implanted cells on the decellularized tissue (FIG. 3B).

In an embodiment, the bioreactor includes a trachea culture chamber (dimensions: 2.5 cm×2.5 cm×1.5 cm; total volume: 9.4 mL) in which the small airways (e.g., rat trachea, human small airways) can be placed for de-epithelialization, cell delivery, and subsequent ex vivo culture (FIG. 1B). The bioreactor is designed and constructed in a way that the luminal surface of the airway can be treated using different solutions (e.g., decellularization solution, washing solution, cells, culture medium) while the entire airway is submerged in a cell culture medium to maintain viability of the airway tissue during experiments. A transparent sheet is attached to the top of the main chamber to prevent contamination of airway tissue inside the bioreactor chamber.

The imaging platform integrated with a bioreactor made from an optical fiber (diameter: 500 um), a scientific camera, an achromatic doublet, a filter lens, a dual-edge super-resolution, a dichroic mirror, and an objective lens is shown in FIG. 2. For imaging tracheal lumen, the distal end of the optical fiber is inserted into the luer connector that is attached to one end of the airway tissue within the bioreactor. The light signals emitted from the luminal surface of the airway is detected at the proximal end of the fiber, which is then visualized via the camera.

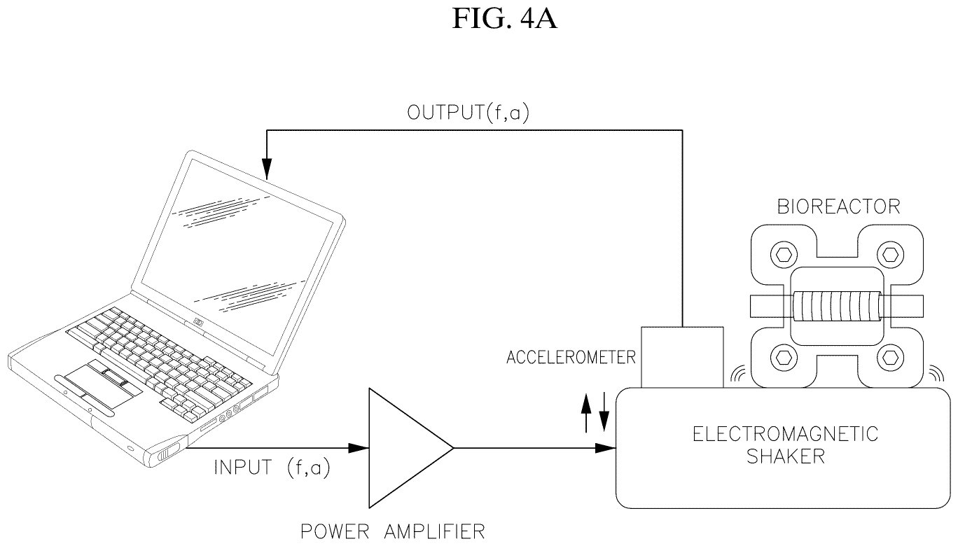

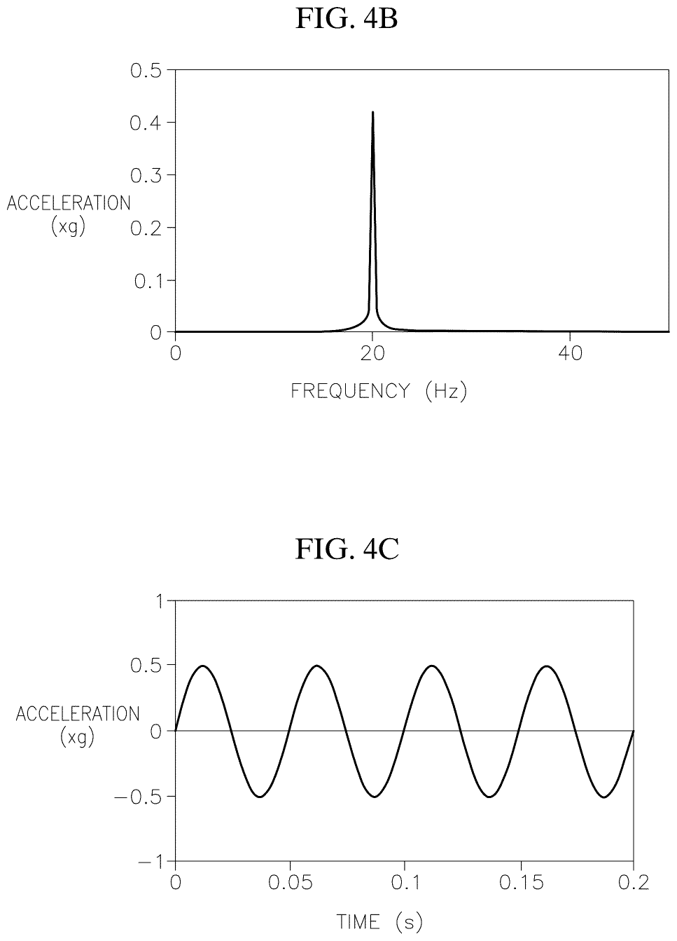

To remove the epithelium from the in vitro-cultured airway, aqueous solution of a detergent (e.g., sodium dodecyl sulfate) is instilled directly into the airway via an inlet cannula. Specifically, a small volume (several microliters) solution can be infused through the airway using a programmable syringe pump to generate a thin film of the detergent solution on the luminal surface of the trachea. To promote removal of cells from the airway lumen, the airway is incubated in the bioreactor at 37′C. The bioreactor is then mechanically vibrated at 20 Hz of frequency using a custom-built shaker while being washed with 1 phosphate buffered saline (1×PBS) solution (FIG. 4). To uniformly distribute the new cells into the de-epithelialized airway, the cell loaded hydrogel is delivered into the airway. The distribution of the cells on the airway lumen can be monitored in real time with optical fiber imaging system integrated with the bioreactor.

### Example 1: In Situ Visualization of the Trachea Lumen

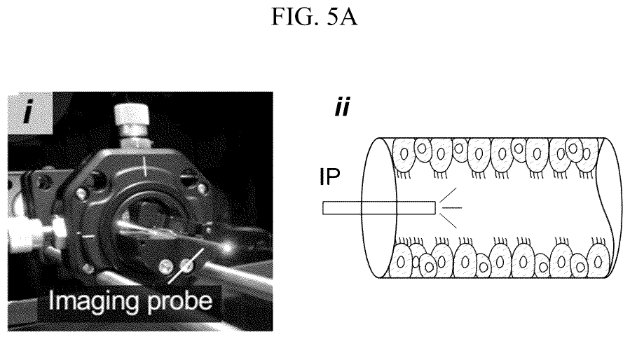

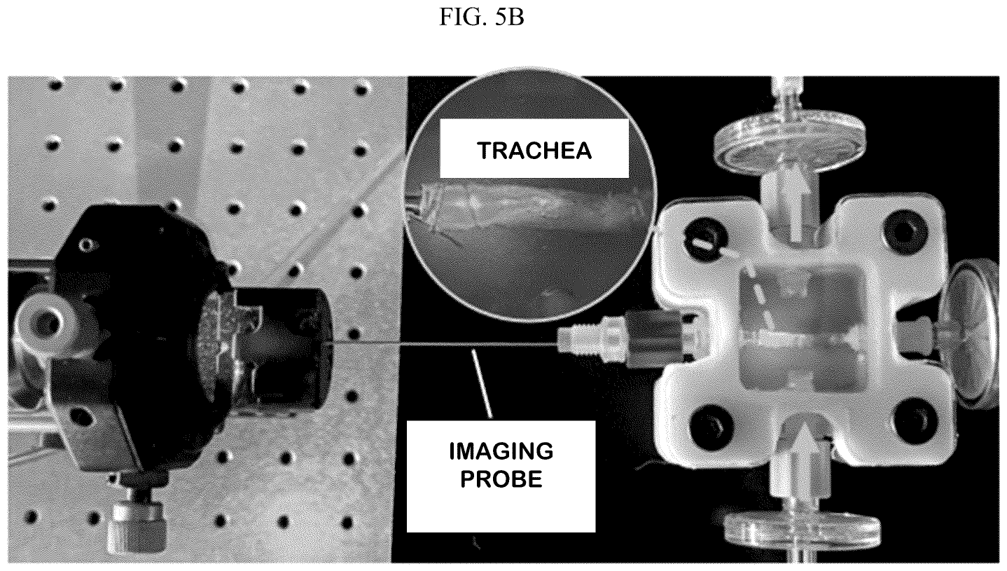

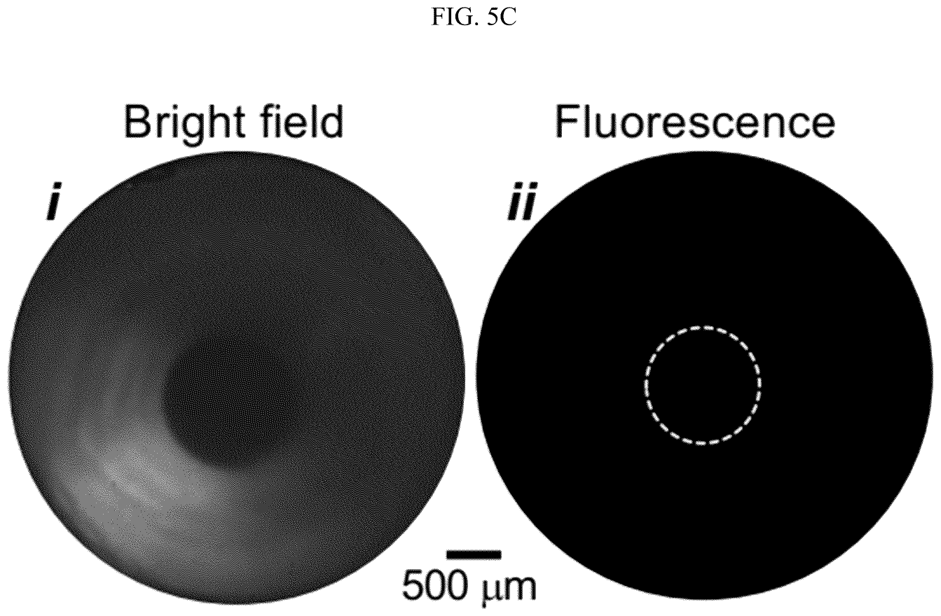

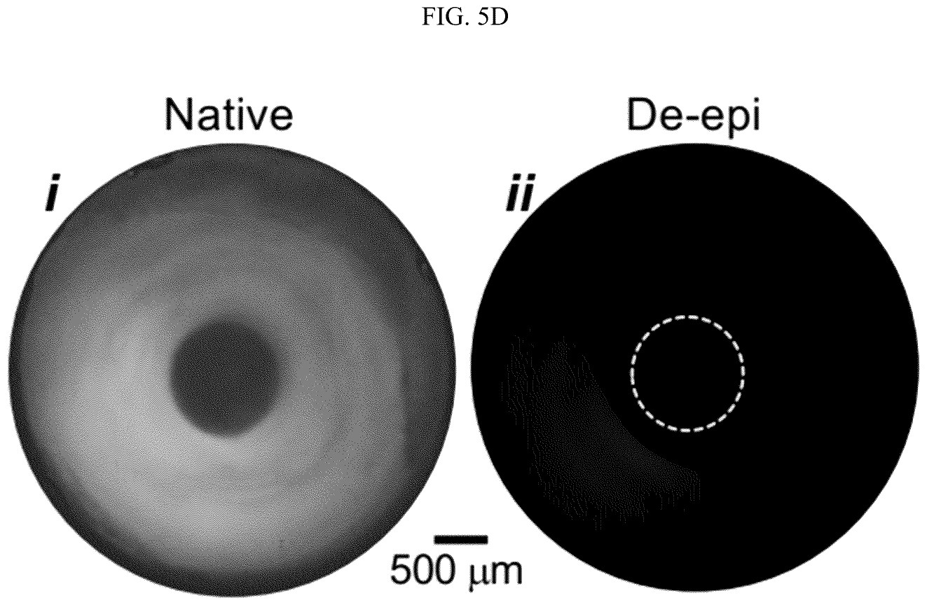

The custom-built in situ airway imaging device was used to inspect the luminal surface of the rat trachea tissue during in vitro cell removal (FIG. 5 A, i-ii). To visualize the trachea lumen, the imaging probe was directly inserted into the trachea through a cannula connected to the trachea (FIG. 5B). Both bright-field (FIG. 5C, i) and fluorescence images (FIG. 5C, ii) of the local luminal surface were obtained, respectively, by using white light and 488-nm laser for illumination prior to carboxyfluorescein succinimidyl ester (CFSE) labeling of the epithelial layer. While no fluorescence signal was observed before labeling, discernible signal (i.e., green light) was observed when the epithelium was labeled with CFSE (FIG. 5D, i). Notably, following de-epithelialization, the intensity of the fluorescent signal substantially decreased, indicating clearance of the epithelium from the tracheal lumen (FIG. 5D, ii). This result highlights the utility of the imaging approach in real-time and of minimally invasive monitoring of the epithelium removal.

### Example 2: Removal of Tracheal Epithelium with Preserved Tissue Extracellular Matrix Components

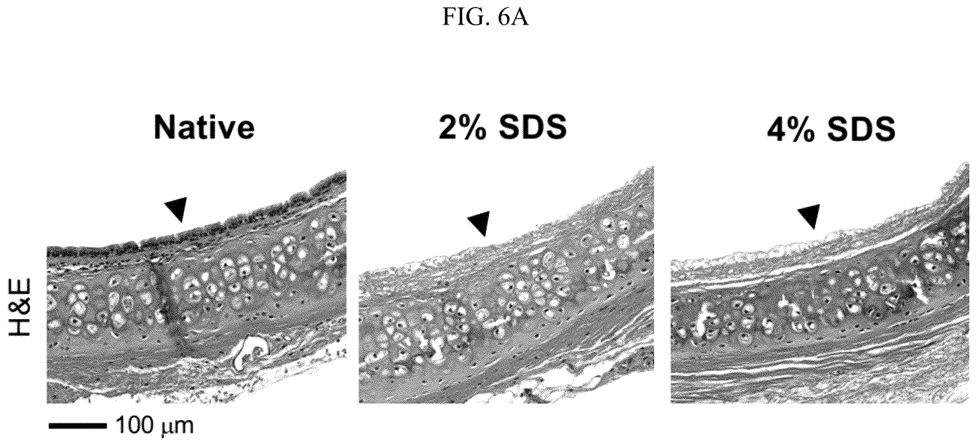

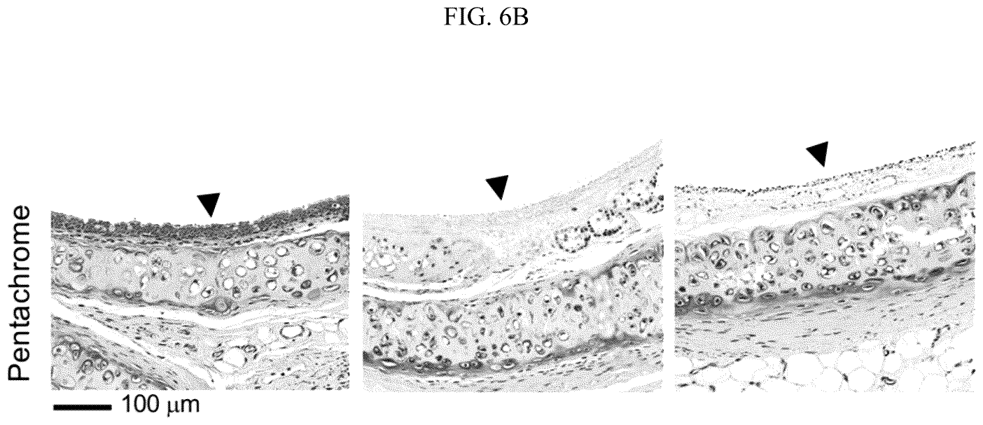

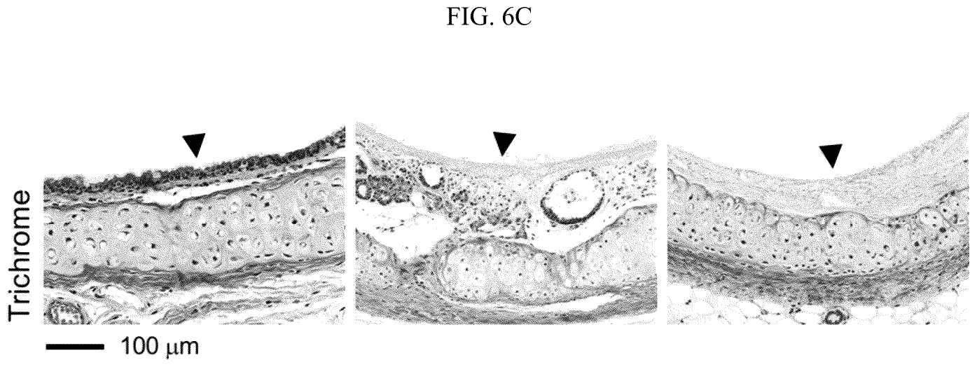

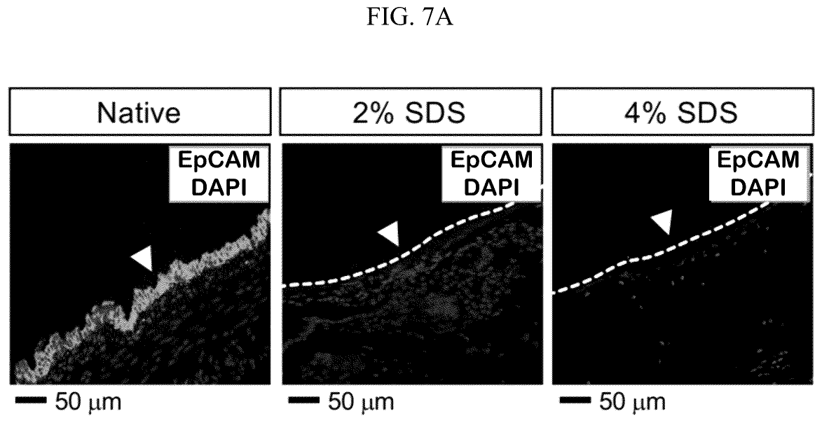

Histological evaluation of de-epithelialized tracheas showed complete removal of the epithelium across the luminal surface of de-epithelialized tracheas that were treated with 2% and 4% detergent solutions (FIG. 6A). In particular, high magnification of H&E images confirmed the removal of the pseudostratified columnar epithelium from the trachea mucosa and preservation of other cells in the submucosa, cartilaginous, and adventitia layers. The structure pattern of the tissue layers and endogenous cells, such as cartilage and chondrocytes, underneath the basement membrane was well preserved following de-epithelialization. Similarly, pentachrome (FIG. 6B) and trichrome (FIG. 6C) staining revealed maintenance of the tissue architecture and extracellular matrix (ECM) components, including collagen and proteoglycans (e.g., mucins), while the airway epithelium was cleared from the lumen.

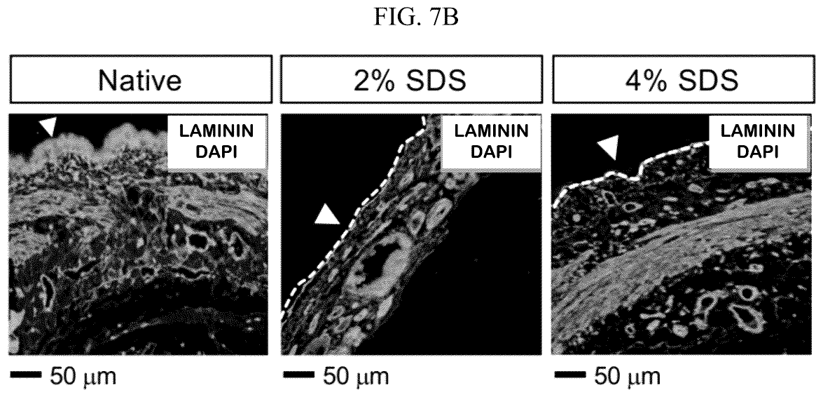

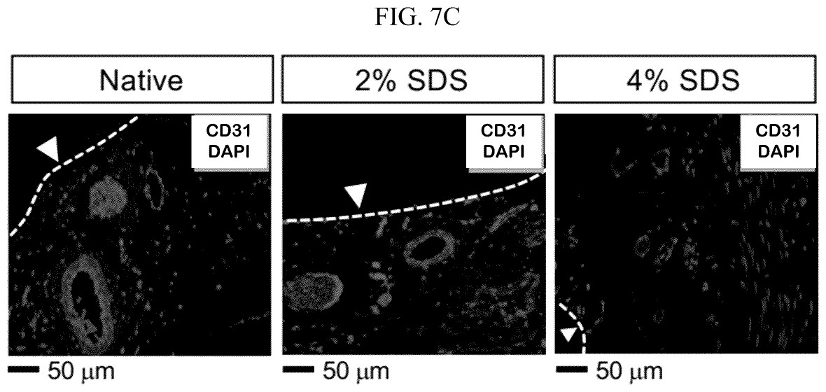

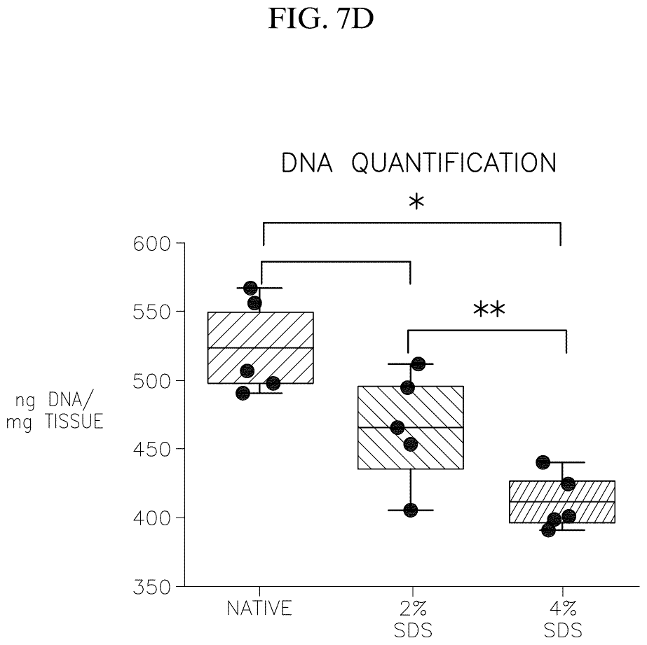

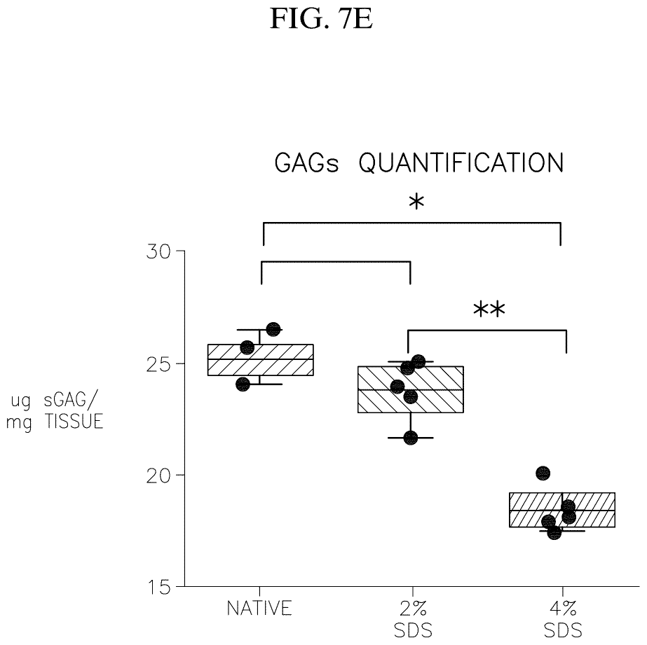

Further, preservation of ECM components of the de-epithelialized tracheas was confirmed via immunofluorescence staining. Immunostaining of the trachea tissues by epithelial cell adhesion molecule (EpCAM) and 4′,6-diamidino-2-phenylindole (DAPI) revealed removal of epithelial layer as no EpCAM (green) and DAPI (blue) signals were detected at the lumen of the trachea (FIG. 7A). De-epithelialized tracheas treated with 4% SDS showed a reduction in the DAPI signal throughout the tissue, suggesting potential damage occurred to endogenous subepithelial cells due to increased detergent concentration.

Nevertheless, the de-epithelialization method preserved laminin (green) (FIG. 7B), immunostaining of the de-epithelialized tissues with endothelial cell marker (cluster of differentiation 31; CD31) confirmed that the blood vessels of the tissue remained intact (FIG. 7C). DNA quantification showed no significant differences between native tissues (0.52+−0.03 μg·mg−1 of tissue) and de-epithelialized tracheas treated with 2% SDS (0.48+−0.03 μg·mg−1 of tissue; p=0.11) (FIG. 7D). On the other hand, the DNA content decreased by nearly 21% in the tracheas treated with 4% SDS (0.41+−0.02 μg·mg−1 of tissue; p<0.001), indicating some of the endogenous cells were affected when the trachea tissues were exposed to higher concentrations of detergent solution. Furthermore, the glycosaminoglycan (GAG) quantification assay (FIG. 7E) revealed no significant difference in sulfated GAG content between native trachea (25.2+−0.9 g·mg−1 tissue) and de-epithelialized tracheas treated with 2% SDS solution (23.8+−1.4 μg·mg−1 of tissue; p=0.17). However, similar to DAPI staining and DNA quantification, nearly 27% of decrease in the sulfated GAGs was observed in the tracheas treated with 4% SDS solution (18.4+−1.0 μg·mg−1 of tissue; p<0.001).

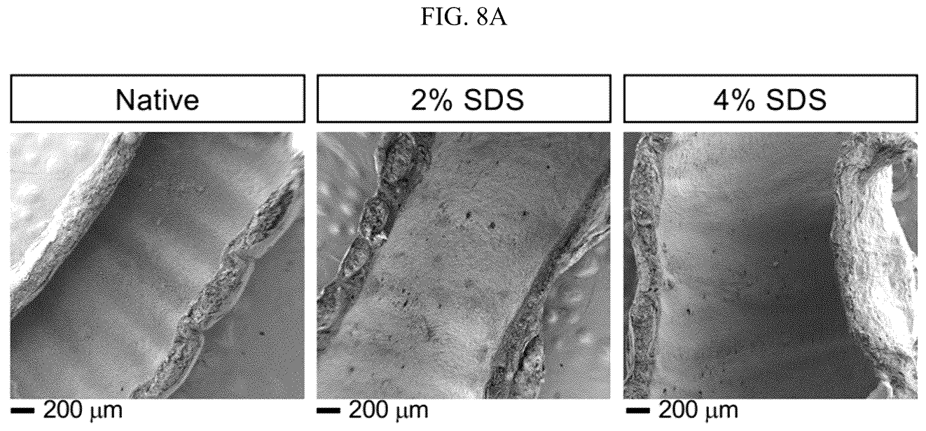

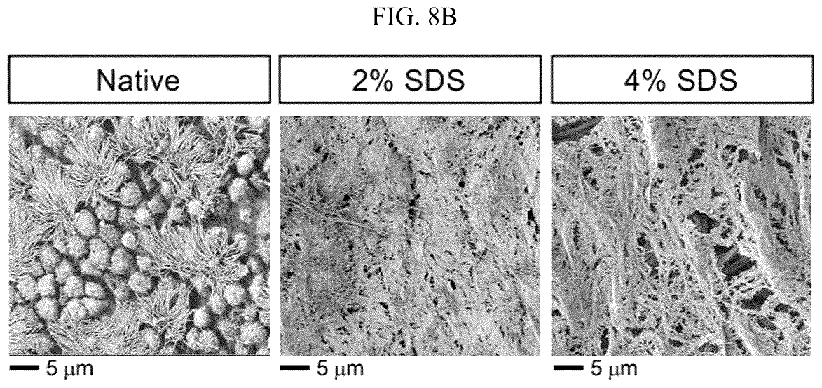

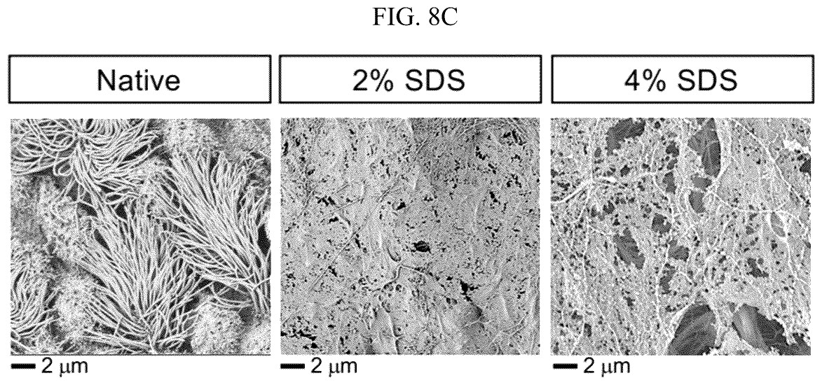

Next, topological changes in the luminal surfaces of de-epithelialized trachea were investigated via SEM imaging where the images were obtained at different magnifications (30× in FIG. 8A; 2000× in FIG. 8B; and 4000× in FIG. 8C). The native trachea showed that the luminal surface was densely populated by different epithelial cells, predominantly multi-ciliated cells, and goblet cells. On the other hand, the SEM images of deepithelialized tracheas with 2% and 4% SDS showed an absence of the tracheal epithelium.

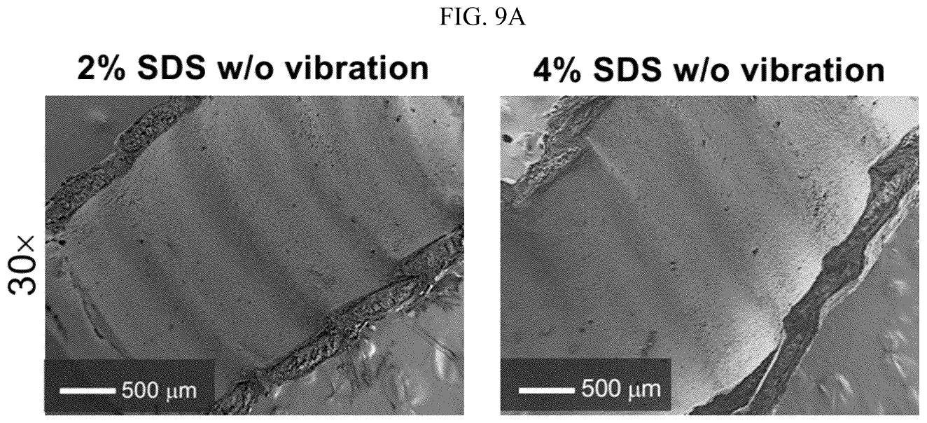

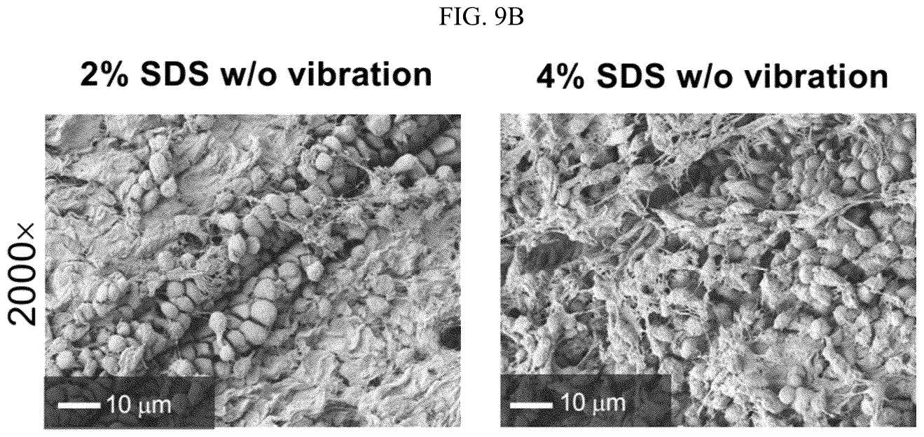

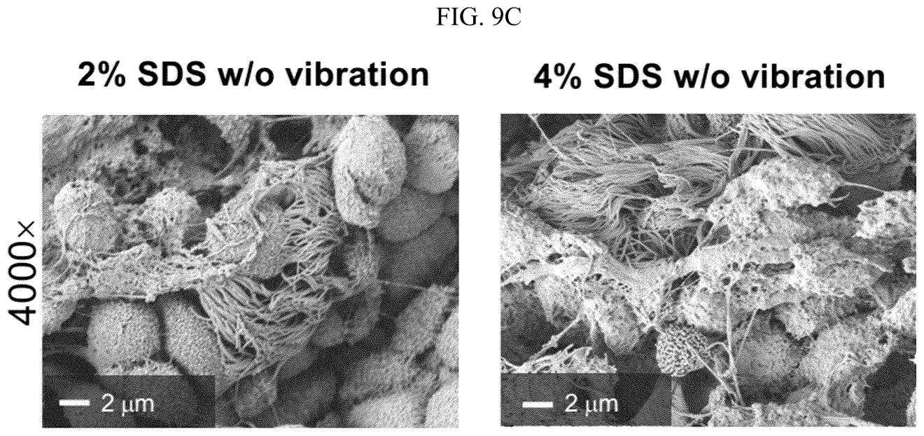

Notably, in both de-epithelialized trachea lumen surfaces, a thin membrane layer, which is most likely the basement membrane, and mesh network of airway ECM were clearly visible. Structural disruption of the basement membrane and tissue ECM was more prominent in the tracheas treated with 4% SDS compared with that of 2% SDS as the porosity of the remaining ECM structure increased with the concentration of the SDS. Notably, use of mechanical vibration during the airway washing facilitated detachment of the epithelium, as SDS disrupted the epithelium remaining attached onto the lumen surface when no vibration was applied to the tissue (FIG. 9). This result clearly indicated that oscillation energy provided to the trachea in the presence of the shear flow promoted disruption and detachment of the detergent-lysed cells from the airway tissue ECM

### Example 3: Homogeneous Distribution of Exogenous Cells onto De-Epithelialized Rat Trachea

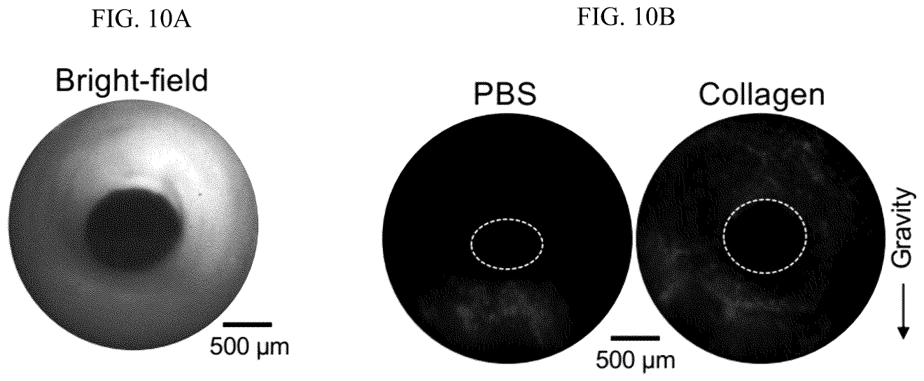

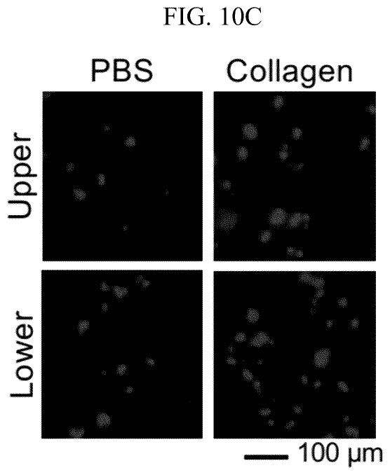

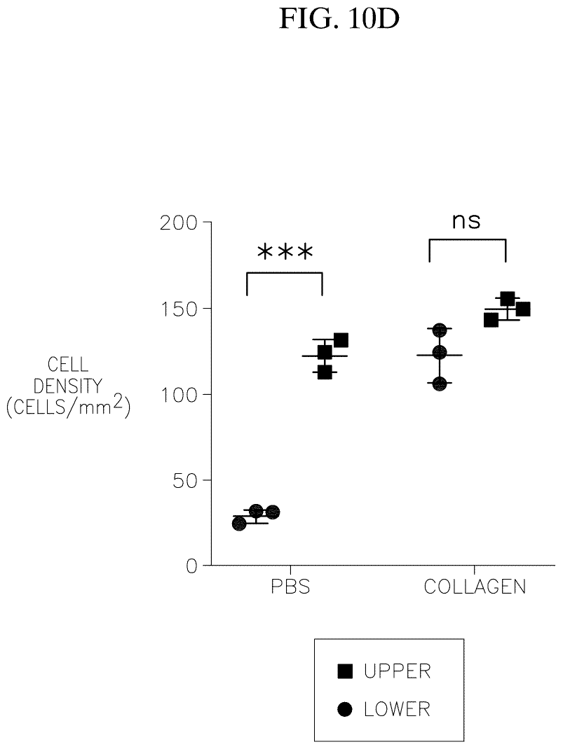

Using ex vivo rat tracheas and fluorescently labeled mesenchymal stem cells (MSCs), it was investigated whether collagen pre-gel could promote homogeneous cell distribution onto the de-epithelialized tracheal lumen (FIG. 10). As in vitro experiments showed that the collagen pre-gel with 3 mg/mL of concentration provided the most uniform particle distribution, that collagen concentration was used in this study. MSCs were labeled with quantum dots and suspended in culture medium (cell concentration: 5×106 cells/mL). Next, 10 μL of the MSC-loaded collagen was instilled into a de-epithelialized rat trachea at 5 mL/min of flow rate and the distribution of the cells on the tracheal lumen was monitored using the optical fiber imaging system (FIG. 5). In bright-field imaging mode, the imaging system clearly showed the interior of the rat trachea (FIG. 10A). Consistent with the in vitro study, the fluorescently labeled cells that were delivered via collagen remained adhered more uniformly across the lumen compared with those seeded via PBS (control; FIG. 10B). Further, the difference of cell seeding density between the upper (123 cells/mm2) and lower half of the tube (150 cells/mm2) was insignificant, highlighting the effectiveness of the collagen-based cell seeding (FIGS. 10C, 10D).

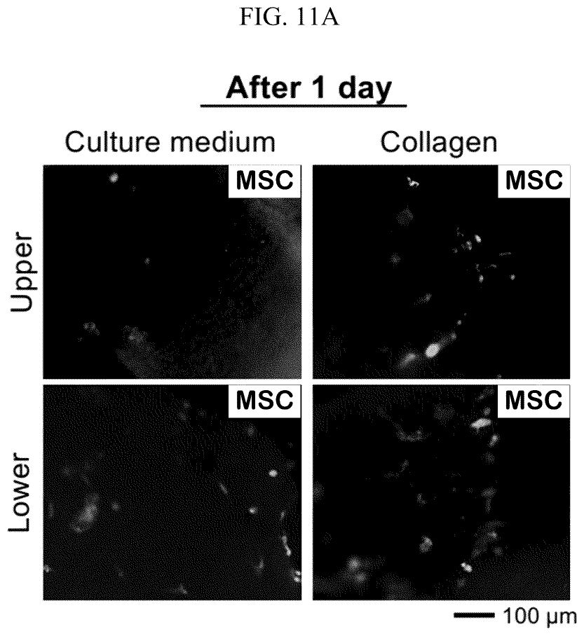

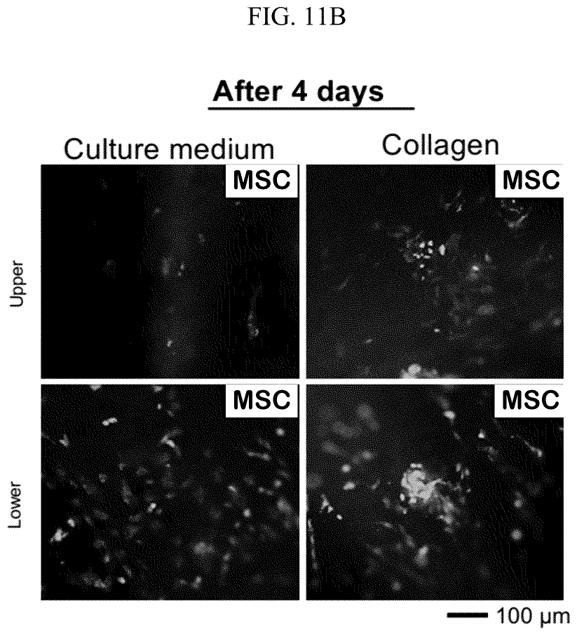

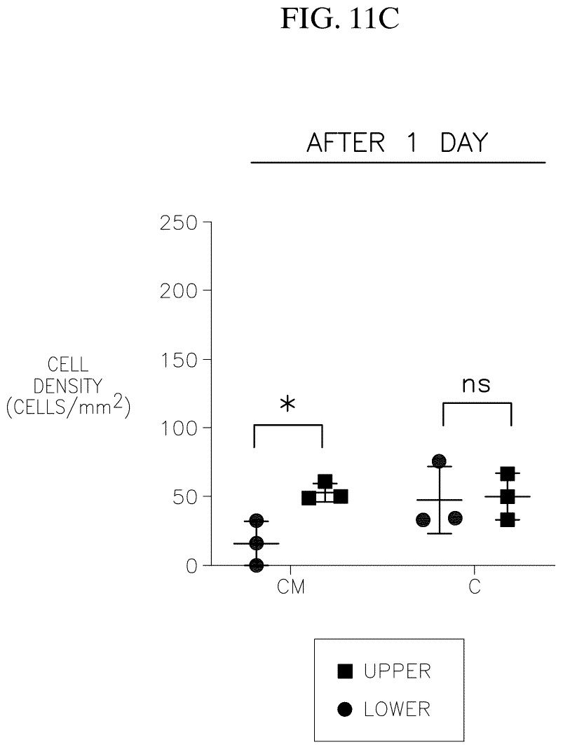

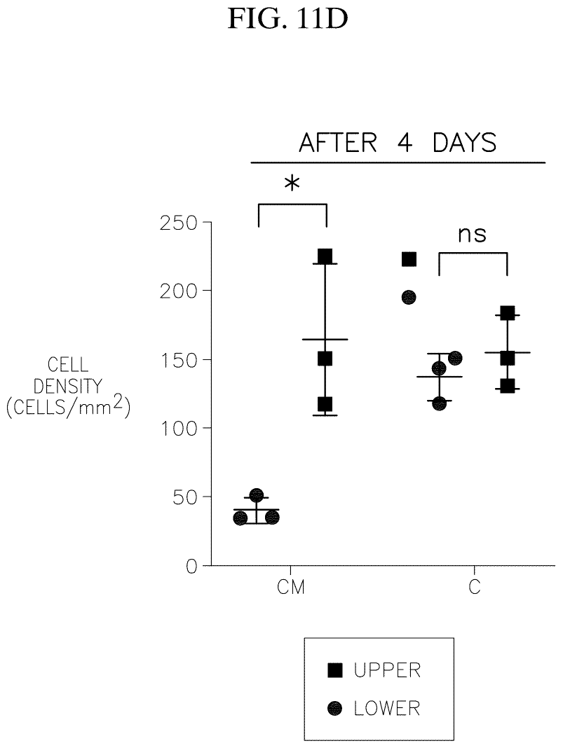

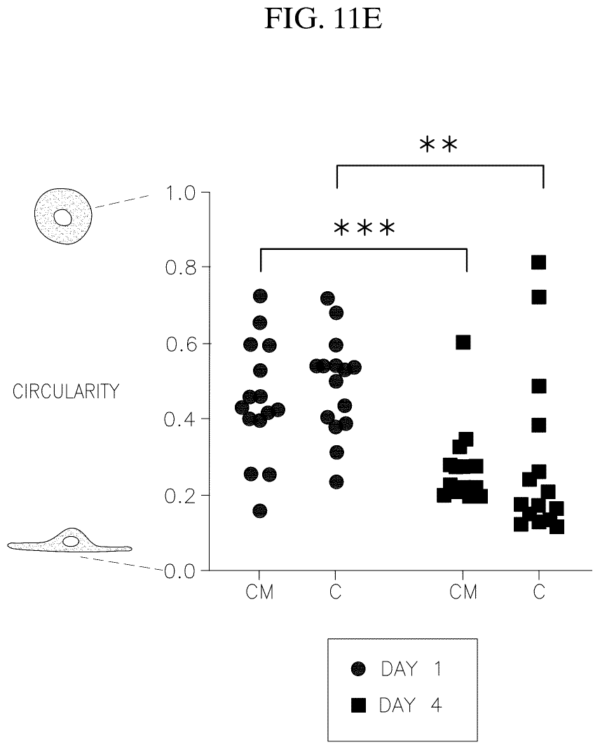

Next, it was investigated whether the cells implanted by collagen pre-gel solution could maintain the homogeneous cell distribution during subsequent ex vivo tissue cultivation, and whether the seeded cells survive and proliferate on the de-epithelialized rat tracheal lumen (FIG. 11). To improve visibility of the cells, MSCs were labeled with carboxyfluorescein succinimidyl ester (CFSE) prior to cell seeding. The bioreactor containing de-epithelialized rat trachea seeded with MSCs was stored in a cell culture incubator up to 4 days. Spatial distributions and shapes of the seeded cells were inspected via fluorescent microscopy following 1 day and 4 days of in vitro cultivation of the cell-tissue constructs. The cells seeded via both collagen and culture medium (control) proliferated on the de-epithelialized trachea surface as indicated by the increased number of cells expressing CFSE overtime in both upper and lower half of the tracheal lumen (FIGS. 11A, 11B). In the collagen group, however, the difference in the density of the cells between upper and lower lumens was minimal after 4 days of in vitro cultivation (upper surface: 137 cells/mm2, lower surface: 154 cells/mm2) (FIGS. 11C, 11D). Meanwhile, cell circularity, which is the ratio of area to perimeter of the cells, was determined to quantitatively evaluate cell engraftment onto the tissue surface (FIG. 11E). In both the collagen and culture medium groups, the seeded cells displayed squamous morphology and reduced circularity at day 4, suggesting that the de-epithelialized tracheas were capable of supporting cell engraftment and survival, and the collagen hydrogel did not alter motility of the implanted cells.

Further embodiments and details relating to the present invention can be found in the aforementioned provisional application and in the manuscripts entitled “Imaging-Guided Bioreactor for De-Epithelialization and Long-Term Cultivation of Ex Vivo Rat Trachea”, “Homogeneous Distribution of Exogeneous Cells onto De-Epithelialized Rat Trachea via Instillation of Cell-Loaded Hydrogel”, and “Imaging-Guided Bioreactor for Generating Bioengineered Airway Tissue” which are attached thereto as Appendices A and B, and C, respectively, the entire contents of all of which are incorporated herein by reference and made a part of the present application for all purposes. Further embodiments and details relating to the present invention can also be found in the posters entitled “Imaging-Enabled Bioreactor for Ex Vivo culture of De-Epithelialized Rat Trachea” and “Selective Replacement Of The Airway Epithelium In In Vitro-cultured Rat Trachea” and two abstracts bearing the same names, which are thereto as Appendices D, E, F, and G, respectively, the entire contents of all of which are also incorporated herein by reference and made a part of the present application for all purposes.

It will be understood that the embodiments described herein are merely exemplary and that a person skilled in the art may make many variations and modifications without departing from the spirit and scope of the invention. All such variations and modifications are intended to be included within the scope of the invention.

1. A bioreactor, adapted for visualizing a tissue sample, comprising:
a culture chamber adapted to receive a tissue sample;
means for supplying a cell culture medium to said culture chamber so as to immerse a tissue sample received in said culture chamber; and
a fiber-optic imaging element integrated with said culture chamber and adapted to visualize said culture chamber under in situ conditions.

2. The bioreactor claim 1, wherein said culture chamber is adapted to receive a tissue sample having a tubular configuration.

3. The bioreactor of claim 2, wherein said culture chamber is adapted to receive a lung tissue sample.

4. The bioreactor of claim 1, wherein said fiber-optic imaging element comprises: a scientific camera; an achromatic doublet; a filter lens; a dual-edge super-resolution; a dichroic mirror; and an objective lens.

5. The bioreactor of claim 1, further comprising an electromagnetic shaker.

6. The bioreactor of claim 1, wherein said bioreactor is adapted to test drug candidates on a tissue sample received in said culture chamber.

7. The bioreactor of claim 1, wherein said bioreactor is integrated on a microchip.

8. The bioreactor of claim 1, wherein said fiber-optic imagining element is adapted to visualize, in real-time, a tissue sample received in said culture chamber.

9. The bioreactor of claim 1, wherein said fiber-optic imaging element is adapted to visualize, in both bright-field visualization and fluorescent visualization, a tissue sample received in said culture chamber.

10. A method for deepithelializing a tissue sample, comprising:
immersing said tissue sample in a cell culture medium;
interfacing said tissue sample with an aqueous solution of detergent;
mechanically vibrating said tissue sample;
washing said tissue sample with a washing solution;
delivering a cell loaded hydrogel to said tissue sample; and
growing new cells on said tissue sample.

11. The method of claim 10, further comprising the step of visualizing said tissue sample.

12. The method of claim 10, wherein said growing step is conducted to grow new cells in a homogeneous distribution.

13. The method of claim 10, wherein said tissue sample is lung tissue.

14. The method of claim 13, wherein said tissue sample is epithelium tissue from a rat.

15. The method of claim 10, wherein said new cells are human cells, and wherein said tissue sample is from a non-human organism.

16. The method of claim 10, wherein said detergent comprises sodium dodecyl sulfate.

17. The method of claim 10, wherein said vibrating step is conducted at 20 Hz.

18. The method of claim 10, wherein said washing solution comprises 1 phosphate buffered saline.

19. The method of claim 10, wherein said cell loaded hydrogel comprises a collagen pre-gel.

20. The method of claim 19, wherein said collagen pre-gel has a concentration of 3 mg/mL.
